(19) United States
(12) Patent Application Publication
(10) Pub. No.: US 2010/0112604 A1
(43) Pub. Date: May 6, 2010

(54) HOME FOOD TEST KIT AND METHOD OF USE

(76) Inventor: Joy Ann Drawdy, Gainesville, FL (US)

Correspondence Address: DAVID L. KING, SR., 5131 N.E. COUNTY ROAD 340, HIGH SPRINGS, FL, 32643, US

(21) Appl. No.: 12/264,438

(22) Filed: Nov. 4, 2008

(51) Int. Cl. G01N 33/542; G01N 31/22

(52) U.S. Cl. 435/7.72; 422/56; 435/7.9

(57) ABSTRACT

A home test kit for detecting the presence or absence of melamine (1,3,5-triazine-2,4,6-triamine) has a sample extract container in which a volume of extract solution is present; a test strip, the test strip being a lateral flow device for detecting melamine (1,3,5-triazine-2,4,6-triamine) in a food or beverage sample; and wherein a food or beverage sample is placed in the sample extract container to form an aqueous sample extract solution and the test strip is inserted directly into the aqueous sample for an instructed time then the strip is removed and observed for a color change. The method of using the kit involves a visual observation without requiring any other equipment.

### TECHNICAL FIELD

This invention relates to a test kit for detecting toxins formed by the use of melamine ingredients in food products for human or pet consumption, more particularly a complete home test kit for use by consumers in the absence of sophisticated equipment or laboratory analysis and that is designed for economical manufacturing to ensure the end product is affordable to a wide audience of consumers.

### BACKGROUND OF THE INVENTION

Control of ingredients used in the manufacture of food products for human or pet consumption has relied on industry compliance and government regulatory agencies.

Within particular regions these standards are rigorously maintained, while in other locations the controls are lacking and often ignored. This creates a risk to people and pets not only within the region producing the food products, but also to any location that imports these food products for consumption.

Emerging food exporting markets such as China and India among others value low cost manufacturing and have been found to use substitute low cost filler ingredients to further reduce cost and increase profits. In processed foods the ability to detect this practice is often costly and, in some parts of the world, is currently unattainable.

In dairy products and other food products a composition of melamine is added to the food product. Melamine enables the food product to mimic high protein pure dairy products, but in fact has no nutritional value. This practice was discovered in baby formula, white chocolates and dairy creamers all originally produced in China. Naturally diluting infant formula in and of itself is simply wrong in that infants are most in need of proper nutrition and the harm caused is irreparable. The problem is more severe in that the filler product, melamine, combines easily and quickly with acids in particular cyanuric acid (2,4,6 trihydroxy-1,3,5 triazine) to create a lethal toxin unfit for consumption. This by-product forms in aged or less refined melamine and when consumed the kidney rapidly produces kidney stones and can lead to kidney failure. In China thousands of infants were exposed to this toxin, several children died in a rather short time, and others have degraded liver and kidney functions that will never recover back to normal.

In the United States, the problem first exposed itself in pet food. When across the pet food industry millions of dogs and cats were exposed to harmful levels of melamine in pet food. Thousands of animals died and many more were so damaged they had to be euthanized. The problem has gotten so bad the Chinese government has asked for new rapid testing procedures and interest in Melamine detection methods has sky rocketed.

The current state of food testing relies on laboratories taking samples and conducting tests using costly and complex equipment. The test equipment employed often includes gas chromatography, mass spectrometers or spectrophotometer analyzers each costing up to several hundreds of thousands of dollars. Although many laboratories offer these testing services to the public, a single sample tested for the presence of melamine is time consuming and often cost prohibitive for the average consumer. More importantly by the time a food product recall can be announced large amounts of the toxic products have been consumed and irreparable damage has already occurred. The problem is this use of laboratories has been ineffective in stopping the trafficking of such dangerous food products.

It is an objective of the present invention to provide a home test kit for the detection of melamine in consumable food and beverage products. This home test kit can be conducted by consumers without requiring any expensive equipment and/or special laboratory training.

### SUMMARY OF THE INVENTION

This invention is a system, protocol, method and apparatus to enable a person unskilled in chemical analysis and without the use of laboratory equipment, to test food and beverage products for the presence or absence of the chemical Melamine.

The invention includes a pre-assembled comprehensive home test kit to be used for testing raw or finished food or beverage products that may be contaminated with Melamine. The home test kit, including simple, safe and disposable materials, is designed to be affordable, non-hazardous, straightforward and easy to use.

Consumers worldwide have long wanted the means to test, at home, the foods they purchase for themselves, their families and their pets to ensure that it has not been contaminated with Melamine. Prior to this invention, no one has conceived a way to provide consumers with this home-test kit which is dependent on an immunochromatographic assay for Melamine that can be read visually and that can be economical to commercially produce for home use.

It seems the principle reasons for this are three-fold: 1) Analytical techniques capable of detecting Melamine in foods or beverages are difficult to modify in a manner that would allow for visual readouts (and at levels low enough to be meaningful to food safety) without the use of sophisticated laboratory equipment such as the spectrophotometer. 2) The type of food and beverage products that have and can be contaminated with Melamine are so multifarious as to make it difficult to create a home-use system of detection that can be used in a variety of products and that does not require complex sample preparation. 3) To be meaningful to the consumer using it, a home test kit for Melamine detection must not only be reliable but also straightforward, easy to use and affordable. While immunochromatographic assay methods are frequently used in home test kits, the use of such a method is problematic when endeavoring to test specifically for Melamine in a home-test format because the variable conditions of samples and intended use require, not just a testing method, but a comprehensive system.

The present invention overcomes the above described and other problems and achieves its primary goal of providing consumers with a rapid, reliable and affordable home test kit for the analysis of foods and beverages that may be contaminated with Melamine.

### BRIEF DESCRIPTION OF THE DRAWINGS

These and further objects of the present invention will be more fully understood from the following description of the invention reference to the illustrations appended hereto:

FIG. 1 is a view of the various components used in the home test kit of the present invention.

FIG. 2 is a cross-sectional view of a lateral flow test strip made in accordance with the present invention.

FIG. 3 is a perspective view of the test strip.

FIG. 3A is an alternative embodiment of the test strip.

FIGS. 4-8 illustrate a proposed method of using the test kit of FIG. 1.

FIG. 4 shows a sample of prepared granules of a food product being inserted in a test vial containing a solution containing a melamine extract solution.

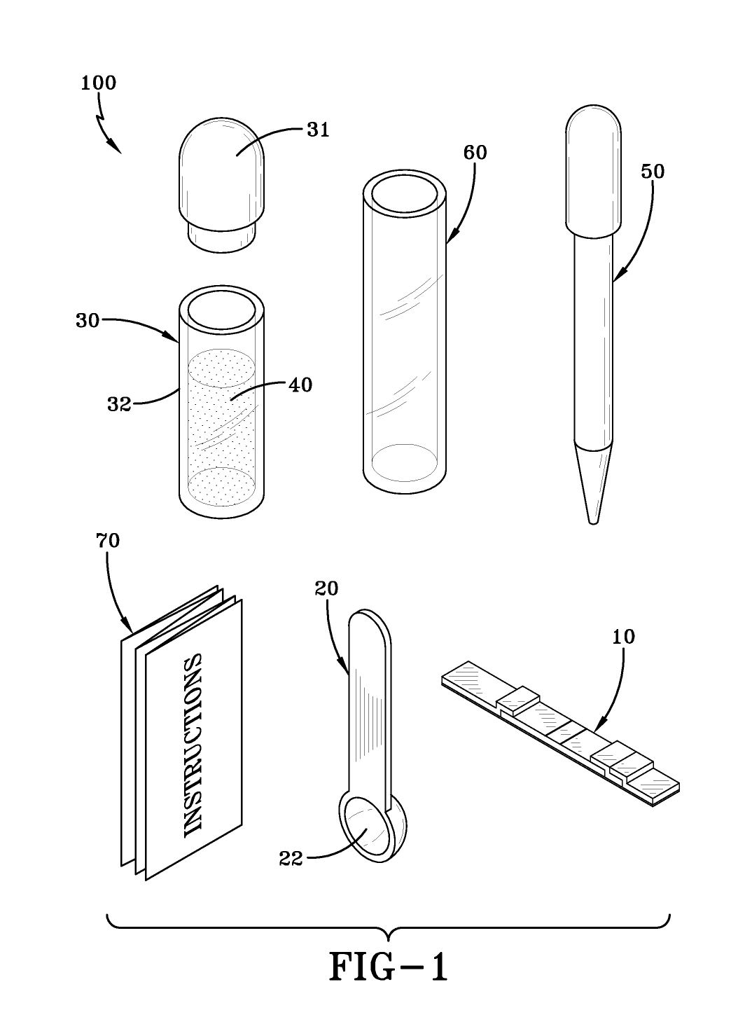

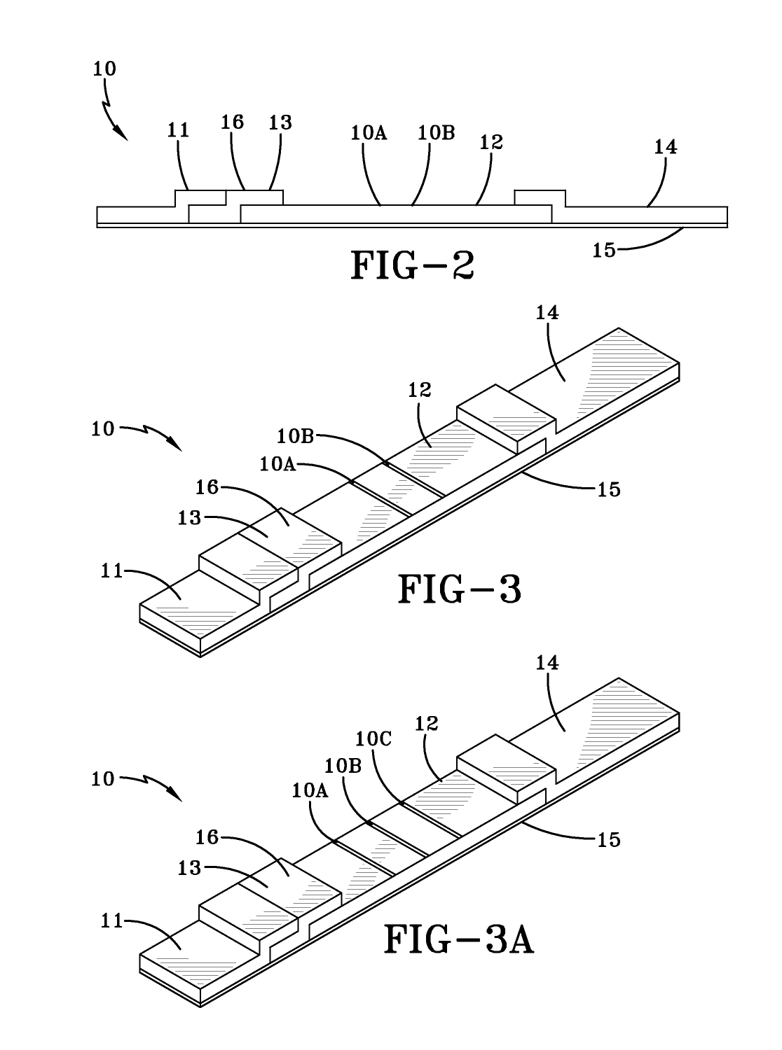

### DETAILED DESCRIPTION OF THE INVENTION

The invention is for a method, device 10 and test kit system 100 that can be used by a person unskilled in the art of analytical chemistry to test a food or beverage product for the presence or absence of Melamine (1,3,5-triazine-2,4,6-triamine).

Method: The preferred method relates to a direct double-antibody immunochromatographic assay using a monoclonal melamine antibody and a melamine horseradish peroxide (HRP) enzyme label with a color tracer reagent such as latex, gold, a colored liposome or colored polymeric bead, tetramethylbenzidine (TMB) or any triarylmethane chromogen that is capable of undergoing a detectable color change upon oxidation with HRP.

Device 10: A lateral flow membrane-based test strip 10 modified to be compatible with the intended use of the test kit 100 and specifically pre-treated for optimal performance of the preferred testing method and to allow for a visual reading indicating the presence or absence of Melamine 2.

Test Kit: Home Test Kit system comprising materials preferred for optimal performance and reliability of the testing method, easy use and straightforward readability of the test results.

The preferred system is a boxed kit 100 comprising a plastic measuring scoop 20 with fill line 22 indicated, sample extract container 30 with a closure or lid 31, preferably the lid or closure 31 is colored blue to match a melamine color indicator, the container cylinder 32 containing Melamine extract solution 40 (such as an acetronitrile/water or methanol/water solution); pipette 50; test tube 60; lateral flow device 10; the lateral flow device being the test strip 10; and brochure insert 70 containing directions for use and other information pertinent to the use of the kit 100.

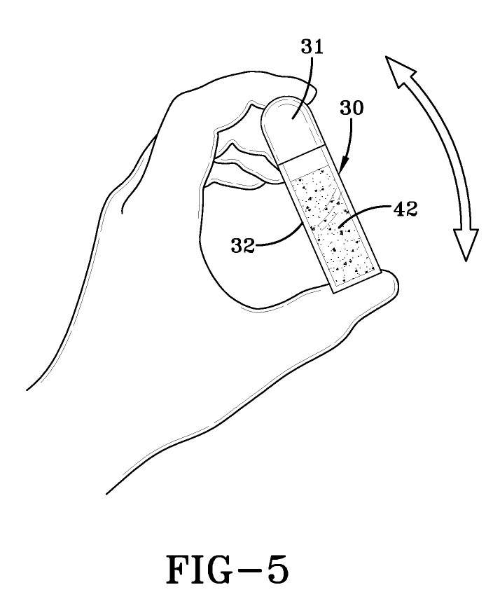

Depending on the consistency of the food or beverage sample 4 (dry, semi-moist or liquid) the user prepares the sample 4 by means of grinding, blending, crushing or simple pouring. Sample is then measured to the fill line indicated on the Measuring Scoop 20. The prepared and measured sample 4 is then added to the Sample Extract Container 30 in which a volume of extraction solution 40 is present as shown in FIG. 4.

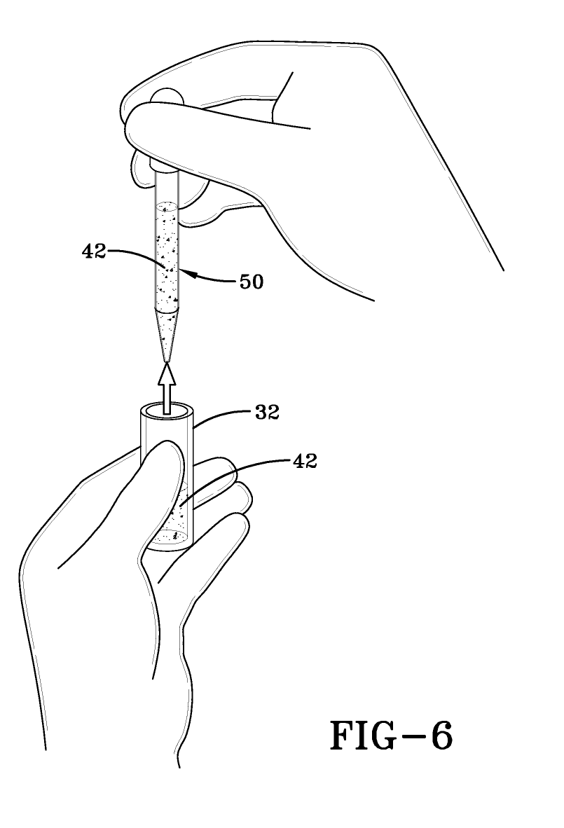

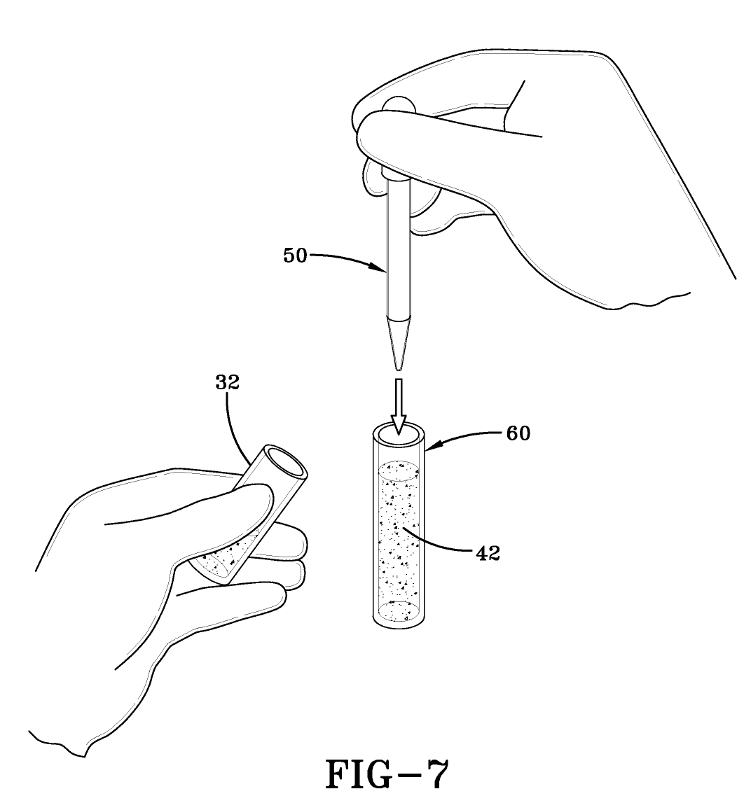

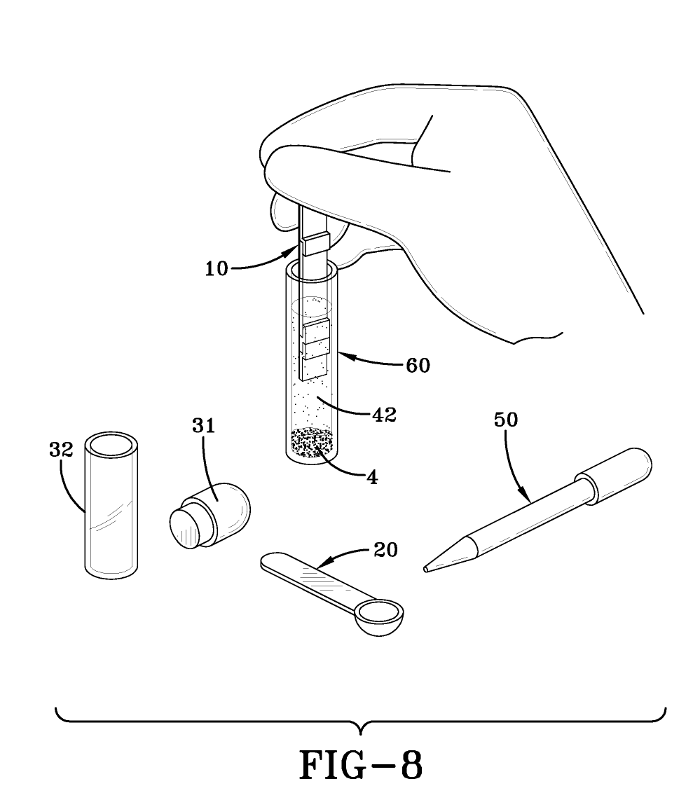

Sample 4 with extraction solution 40 is then shaken/mixed to produce an aqueous sample extract solution 42 as shown in FIG. 5. Using the Pipette 50, user then transfers the entire contents 42 of the Sample Extract Container 30 to the Test Tube 60 component of the kit 100 as shown in FIGS. 6 and 7. The Lateral Flow test strip component 10 of the kit 100 is then inserted into the Test Tube 60 as shown in FIG. 8. Alternately, the test strip 10 can be inserted directly into the mixed sample/extract solution container 30. The user then waits the instructed time of preferably 15 to 60 minutes, more preferably 20 to 30 minutes then removes the test strip 10 and observes for any color changes.

Method and Device: An immunochromatographic assay with a direct (double antibody sandwich) reaction scheme performed within a lateral flow test strip device 10 is preferred. In this scheme, a lateral flow test strip device 10 has a nitrocellulose (NC) membrane 12, sample pad “dipstick” 11, conjugate pad 13, absorbent pad and plastic adhesive backing card 15 is used. Antibody specific to Melamine such as mouse monoclonal antibodies to Triazines is sprayed and immobilized in a test line 10A on the surface of the NC membrane. As a Detection Conjugate 16, a second antibody reagent such as horseradish peroxide (HRP) enzyme, also recognizing Melamine and labeled with a color tracer reagent is contained within the conjugate pad 13 on the membrane 12.

Alternate Version of the strip 10 may use an Antibody specific to Melamine sprayed and immobilized in a test line 10A on the surface of the NC membrane 12. As a Detection Conjugate 16, a second antibody reagent, also recognizing Melamine and labeled with a color tracer reagent is contained within the conjugate pad 13 on the membrane 12.

When applied to the Sample Pad 14, liquid from the sample extract 42 moves by capillary action into the conjugate pad 13 and re-hydrates the detection conjugate 16. There, the detection conjugate 16 specifically binds to Melamine and the complex of sample extract 42 and detection conjugate 16 moves into and up the membrane 12. The sample passes through the test line 10A where the immobilized Melamine Antibody captures the Melamine-antibody-substrate complex forming an antibody sandwich. If the sample 42 is positive for the presence of Melamine 2, the test line 10A develops a color and if no melamine 2 is present in the sample 42, no antibody-melamine sandwich is formed and the test line 10A does not turn a color. Immobilized reagents at the control line capture excess color reagent that has passed through the test line.

This causes the control line 10B to develop a color which indicates that the test was performed correctly. The user can then visually read the results of the test. A single control line 10B on the NC membrane 12 indicates the test was performed correctly and that the sample 42 tested negative. Two lines on the NC Membrane 12 indicate that the test was performed correctly and that the sample 42 tested positive meaning melamine was detected.

Foodstuffs can sometimes be contaminated with remarkably high doses of melamine. For instance, an October 2008 report revealed that one Chinese company sold infant formula spiked with Melamine at a level of 6,196 parts per million. This is an incredible 2,500 times higher than the FDA safe limit.

This can present a problem with detection because very high doses of Melamine 2 in a sample 4 can create a condition in which unbound Melamine can block the test line 10A and interfere with the labeled-antibody-bound antigen, resulting in a “hook effect” or false negative.

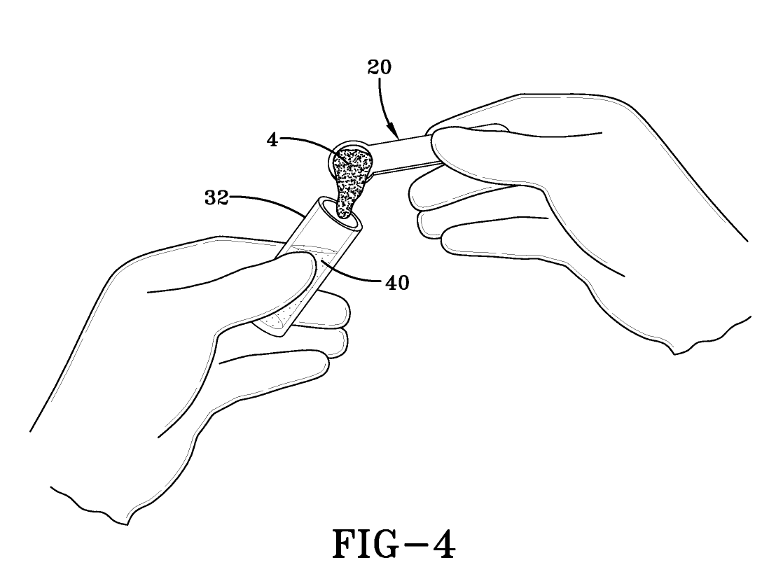

This problem is solved by adding a “hook line” 10C to the test strip 10 as shown in FIG. 3A. A third capture line 10C can be immobilized on the surface of nitrocellulose membrane 12 using a binding partner for the labeled antibody which will bind labeled antibody that did not form a complex with the sample 42 and facilitate its capture on that line 10C.

This allows the user to determine if a negative or weak result is due to low levels of Melamine 2 in the sample 4 or to excessively high levels. When the user reads the test, if the results are weak or negative and the Hook Line is present, that means the results are weak or negative. If the results are weak or negative and the Hook Line is NOT present, this indicates a “hook effect” or very high levels of Melamine 2 in the sample 4.

If a sample tests positive or if a high dose effect is indicated, the user should clearly not use the food of beverage for consumption. Such a test result could be used to alert FDA or any responsible authorities to retest the product for a possible recall. The advantage of the test kit is consumers can assist regulatory agencies in monitoring human and pet food supplies while using the test results themselves to establish a “first defense” determination of safety for immediate home use of a suspect food or beverage.

Alternatively, it is feasible to create new antibodies that specifically bind to Melamine and that can serve to detect Melamine in the present invention.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

1. A lateral flow device found as a test strip for detecting melamine (1,3,5-triazine-2,4,6-triamine) in a food or beverage sample comprises:
a backing card;
a membrane;
a sample pad;
a conjugate pad;
an absorbent pad; and
wherein a first antibody specific to melamine is sprayed and immobilized in a test line on a surface of the membrane, and a second antibody reagent also recognizing melamine and labeled with a color trace reagent is contained in the conjugate pad on the membrane.

2. The device of claim 1 wherein the membrane is made of nitrocellulose (NC).

3. The device of claim 1 wherein the first antibody is mouse monoclonal antibodies to triazines.

4. The device of claim 1 wherein the second antibody reagent is a horseradish peroxide (HRP) enzyme.

5. The device of claim 1 further comprises:
a hook line, the hook line being a third capture line immobilized on the surface of the membrane having a binding partner for labeled antibody which will bind the labeled antibody that did not form a complex with the test sample and facilitate its capture on the hook line.

6. The device of claim 4 wherein the horseradish peroxide (HRP) enzyme label further includes a label with a color tracer reagent.

7. The device of claim 6 wherein the color tracer label is selected from a group including latex, gold, a colored liposome or colored polymeric bead, tetramethylbenzidine (TMB) or any triarylmethane chromogen that is capable of undergoing a detectable color change upon oxidation with HRP.

8. A home test kit for detecting the presence or absence of melamine (1,3,5-triazine-2,4,6-triamine) comprises:
a sample extract container in which a volume of extract solution is present;
a test strip, the test strip being a lateral flow device for detecting melamine (1,3,5-triazine-2,4,6-triamine) in a food or beverage sample; and
wherein a food or beverage sample is placed in the sample extract container to form an aqueous sample extract solution and the test strip is inserted directly into the aqueous sample for an instructed time then the strip is removed and observed for a color change.

9. The home test kit of claim 8 further comprises:
a pipette; and
a test tube, wherein the aqueous sample extract solution is pipetted from the sample extract container and transferred into the test tube and the test strip placed in the test tube containing the aqueous sample extract solution for the instructed time then the test strip is removed and observed for a color change.

10. The home test kit of claim 8 further comprises:
a sample scoop with a fill line for measuring a sample size.

11. The home test kit of claim 8 wherein the test strip comprises:
a backing card;
a membrane;
a sample pad;
a conjugate pad;
an absorbent pad; and
wherein a first antibody specific to melamine is sprayed and immobilized in a test line on a surface of the membrane, and a second antibody reagent also recognizing melamine and labeled with a color trace reagent is contained in the conjugate pad on the membrane.

12. The device of claim 11 wherein the membrane is made of nitrocellulose (NC).

13. The device of claim 11 wherein the first antibody is mouse monoclonal antibodies to triazines.

14. The device of claim 11 wherein the second antibody reagent is a horseradish peroxide (HRP) enzyme.

15. The device of claim 11 further comprises:
a hook line, the hook line being a third capture line immobilized on the surface of the membrane having a binding partner for labeled antibody which will bind the labeled antibody that did not form a complex with the test sample and facilitate its capture on the hook line.

16. The device of claim 14 wherein the horseradish peroxide (HRP) enzyme label further includes a label with a color tracer reagent.

17. The device of claim 16 wherein the color tracer label is selected from a group including latex, gold, a colored liposome or colored polymeric bead, tetramethylbenzidine (TMB) or any triarylmethane chromogen that is capable of undergoing a detectable color change upon oxidation with HRP.

18. A method of testing a food or beverage product for the presence or absence of melamine (1,3,5-triazine-2,4,6-triamine) comprises:
preparing a test sample, depending on the consistency of the food or beverage sample 4 (dry, semi-moist or liquid) the user prepares the sample 4 by means of grinding, blending, crushing or simple pouring;
measuring a pre-selected amount of the test sample;
adding the test sample to a sample extract container in which a volume of an extract solution is present;
shaking or mixing the sample with the extract solution to produce an aqueous sample extract solution;
placing a test strip directly into the aqueous sample extract solution;
waiting for an instructed time prior to removing the test strip; and
observing for any color change.
